(19) United States
(12) Patent Application Publication
(10) Pub. No.: US 2022/0152355 A1
(43) Pub. Date: May 19, 2022

(54) CATHETERS HAVING STEERABLE DISTAL PORTIONS, AND ASSOCIATED SYSTEMS AND METHODS

(71) Applicant: Inari Medical, Inc., Irvine, CA (US)

(72) Inventors: Edward Enriquez Dolendo, Mission Viejo, CA (US); Thomas Robert Brodt, Long Beach, CA (US); Thomas M. Tu, Louisville, KY (US)

(21) Appl. No.: 17/529,064

(22) Filed: Nov. 17, 2021

Related U.S. Application Data
(60) Provisional application No. 63/115,515, filed on Nov. 18, 2020.

(51) Int. Cl. A61M 25/01; A61M 25/00

(52) U.S. Cl. CPC A61M 25/0147; A61M 25/0023; A61M 25/005; A61M 25/0138

(57) ABSTRACT

Disclosed herein are clot removal systems including steerable catheters, and associated systems and methods. In some embodiments, a clot removal system includes (i) an aspiration catheter having a proximal region and a distal region and (ii) a handle coupled to the proximal region of the catheter and having an actuator. The distal region of the catheter can include a deflectable member, and the clot removal system can include a pull wire extending between the actuator and the deflectable member. Actuation of the actuator is configured to pull the pull wire to deflect the deflectable member to deflect the distal region relative to the proximal region. The deflection can facilitate steering of the catheter to hard-to-reach (e.g., tortuous) portions of the anatomy of a patient.

### CROSS-REFERENCE TO RELATED APPLICATION

This application claims the benefit of U.S. Provisional Patent Application No. 63/115,515, filed Nov. 18, 2020, and titled “CATHETERS HAVING STEERABLE DISTAL PORTIONS, AND ASSOCIATED SYSTEMS AND METHODS,” which is incorporated herein by reference in its entirety.

### TECHNICAL FIELD

The present technology generally relates to clot removal systems including catheters (e.g., large bore aspiration catheters) having a steerable distal portion to, for example, facilitate positioning of the catheter in hard-to-reach regions of the vasculature of a patient.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. Infarction does not develop or is greatly limited if the flow of blood is reestablished rapidly. Failure to reestablish blood flow can lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT arises most commonly when there is a propensity for stagnated blood (e.g., long distance air travel, immobility, etc.) and clotting (e.g., cancer, recent surgery, such as orthopedic surgery, etc.). DVT causes harm by: (1) obstructing drainage of venous blood from the legs leading to swelling, ulcers, pain, and infection, and (2) serving as a reservoir for blood clots to travel to other parts of the body including the heart, lungs, brain (stroke), abdominal organs, and/or extremities.

In the pulmonary circulation, the undesirable material can cause harm by obstructing pulmonary arteries—a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

There are many existing techniques to reestablish blood flow through an occluded vessel. One common surgical technique, an embolectomy, involves incising a blood vessel and introducing a balloon-tipped device (such as the Fogarty catheter) to the location of the occlusion. The balloon is then inflated at a point beyond the clot and used to translate the obstructing material back to the point of incision. The obstructing material is then removed by the surgeon. Although such surgical techniques have been useful, exposing a patient to surgery may be traumatic and best avoided when possible. Additionally, the use of a Fogarty catheter may be problematic due to the possible risk of damaging the inner lining of the vessel as the catheter is being withdrawn.

Percutaneous methods are also utilized for reestablishing blood flow. A common percutaneous technique is referred to as balloon angioplasty where a balloon-tipped catheter is introduced to a blood vessel (e.g., typically through an introducing catheter). The balloon-tipped catheter is then advanced to the point of the occlusion and inflated to dilate the stenosis. Balloon angioplasty is appropriate for treating vessel stenosis, but it is generally not effective for treating acute thromboembolisms as none of the occlusive material is removed and the vessel will re-stenos after dilation. Another percutaneous technique involves placing a catheter near the clot and infusing streptokinase, urokinase, or other thrombolytic agents to dissolve the clot. Unfortunately, thrombolysis typically takes hours to days to be successful. Additionally, thrombolytic agents can cause hemorrhage and in many patients the agents cannot be used at all.

Various devices exist for performing a thrombectomy or removing other foreign material. However, such devices have been found to have structures which are either highly complex, cause trauma to the treatment vessel, or lack sufficient retaining structure and thus cannot be appropriately fixed against the vessel to perform adequately. Furthermore, many of the devices have highly complex structures that lead to manufacturing and quality control difficulties as well as delivery issues when passing through tortuous or small diameter catheters. Less complex devices may allow the user to pull through the clot, particularly with inexperienced users, and such devices may not completely capture and/or collect all the clot material.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIGS. 1A and 1B are partially schematic side and isometric views, respectively, of a clot removal system in accordance with embodiments of the present technology.

FIG. 2 is an enlarged, partially cut-away side view of a portion of a proximal region of a catheter of the clot removal system of FIGS. 1A and 1B in accordance with embodiments of the present technology.

FIG. 3A is an enlarged, partially cut-away side view, and FIG. 3B is an enlarged, partially cut-away isometric view, of a portion of an intermediate region of the catheter of FIGS. 1A and 1B in accordance with embodiments of the present technology.

FIGS. 4A and 4B are a distally-facing isometric view and an enlarged proximally-facing isometric view, respectively, of a deflectable member of a deflectable region of the catheter of FIGS. 1A and 1B in accordance with embodiments of the present technology.

FIGS. 4C and 4D are isometric views of a proximal ring and a distal ring, respectively, of the deflectable member of FIGS. 4A and 4B in accordance with embodiments of the present technology.

FIG. 5A is a partially cross-sectional side view of a handle and a portion of the proximal region of the catheter of the clot removal system of FIGS. 1A and 1B in accordance with embodiments of the present technology; and FIG. 5B is an enlarged cross-sectional isometric view of a portion of the handle shown in FIG. 5A.

FIGS. 6A and 6B are a distally-facing isometric view and a side view, respectively, of a deflectable member in accordance with additional embodiments of the present technology.

FIG. 6C is top view of a flat pattern that can be cut to integrally form a proximal ring and a tube portion of the deflectable member of FIGS. 6A and 6B in accordance with embodiments of the present technology; and FIG. 6D is an enlarged top view of a portion of the pattern shown in FIG. 6C.

FIGS. 7A-7C are side views of a portion of the catheter of the clot removal system of FIGS. 1A and 1B during a procedure for removing clot material from within a blood vessel of a patient in accordance with embodiments of the present technology.

### DETAILED DESCRIPTION

The present technology is generally directed to clot removal systems including aspiration catheters having a deflectable/steerable distal portion for improved flexibility through hard-to-reach (e.g., tortuous) vascular anatomy of a patient, and associated systems and methods. In some embodiments, a clot removal system in accordance with embodiments of the present technology includes (i) an aspiration catheter having a proximal region and a distal region and (ii) a handle coupled to the proximal region of the catheter and having an actuator. The distal region of the catheter can include a deflectable member, and the clot removal system can include a pull wire extending between the actuator and the deflectable member. Actuation of the actuator is configured to pull the pull wire to deflect the deflectable member to deflect the distal region relative to the proximal region. The deflection can facilitate steering of the catheter to the hard-to-reach portions of the anatomy of the patient.

In some embodiments, the deflectable member includes a proximal ring, a distal ring, and a tube portion extending between the proximal and distal rings. The proximal ring can include an annular member coupled (e.g., welded) thereto and configured to slidably receive the pull wire. The distal ring can be configured to be fixedly attached (e.g., welded) to the pull wire. The tube portion can include a plurality of openings (e.g., circumferentially extending openings) that define a plurality of ribs. The ribs can flex away from each other when the actuator is actuated to pull the pull wire. In some embodiments, the tube portion further includes a spine extending between the proximal and distal rings and generally aligned with the pull wire.

In some embodiments, the catheter further includes an intermediate region between the proximal and distal regions. The catheter can include a braid of wires extending along the proximal and distal regions, and a coil extending over the braid along the intermediate region.

In some aspects of the present technology, the catheter is configured to be steered to and positioned in difficult-to-reach regions of the anatomy of a patient while still having a relatively large size (e.g., 20 French, 24 French, greater than 24 French). More particularly, the catheter can have an improved torque response and flexibility compared to conventional catheters having the same size. For example, the braid can provide good torque response along the proximal and intermediate regions of the catheter. Additionally, the deflectable region can be configured (e.g., shaped, sized) to be positioned within and steered/flexed into the difficult-to-reach regions of the anatomy. Further, the coil can provide increased hoop strength at the intermediate region while still allowing the catheter to flex. For example, the coil can inhibit or even prevent kinking or other unwanted movement of the catheter when the catheter is aspirated during a clot removal procedure.

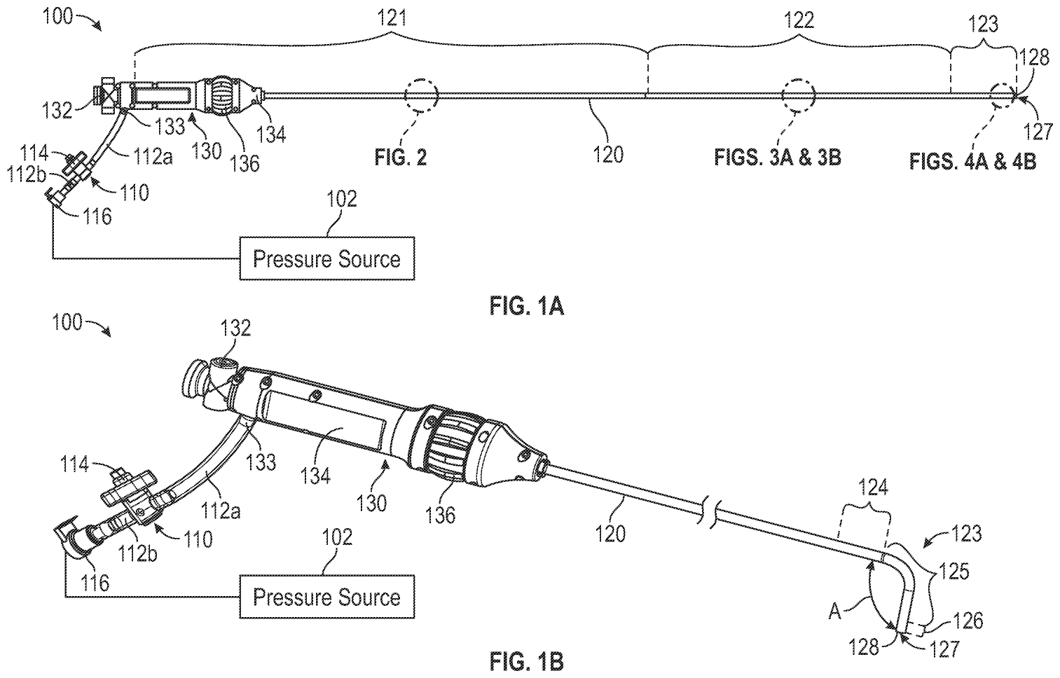

Certain details are set forth in the following description and in FIGS. 1-7C to provide a thorough understanding of various embodiments of the present technology. In other instances, well-known structures, materials, operations, and/or systems often associated with intravascular procedures, clot removal procedures, catheters, and the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures. The systems of the present technology can be used in any orientation suitable to the user.

FIGS. 1A and 1B are partially schematic side and isometric views, respectively, of a clot removal system 100 in accordance with embodiments of the present technology. The clot removal system 100 can also be referred to as an aspiration assembly, a clot treatment system, and/or a thrombectomy system. Referring to FIGS. 1A and 1B together, the clot removal system 100 includes a tubing assembly 110 coupled to a catheter 120 via a handle 130. In general, the clot removal system 100 (i) can include features generally similar or identical to those of the clot removal systems described in detail in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

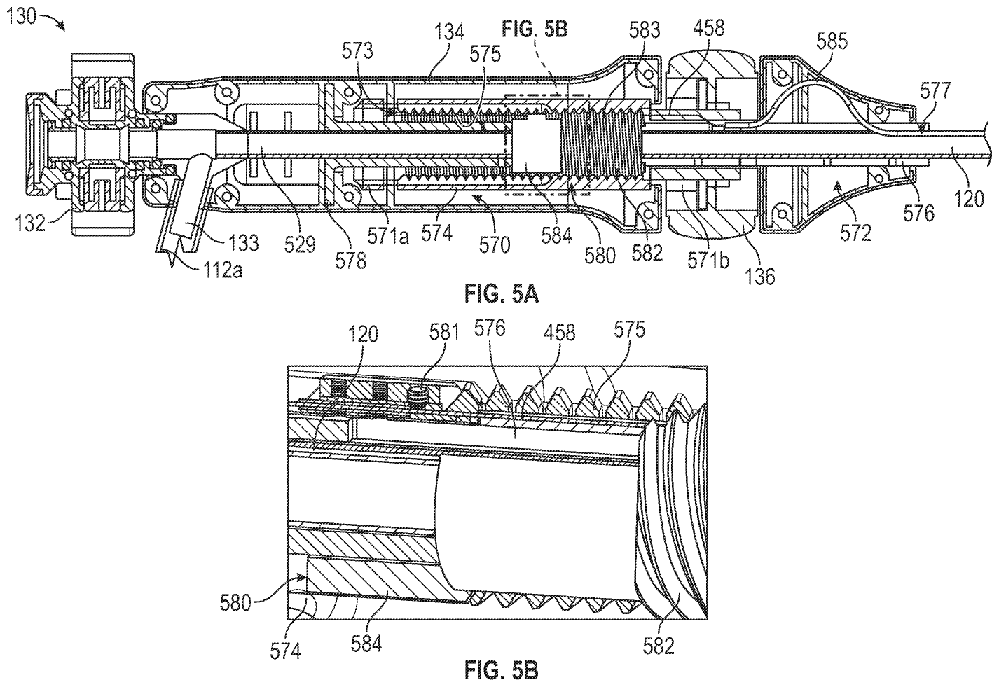

Referring to FIG. 1A, the catheter 120 can include (i) a proximal region or portion 121, (ii) an intermediate region 122 adjacent to and distal of the proximal region 121, and (iii) a distal region 123 adjacent to and distal of the intermediate region 122. Referring to FIG. 1B, the distal region 123 can further include a transition region 124, a deflectable region 125 (e.g., a flexible region, steerable region, deformable region) distal of the transition region 124, and a tip region 126 distal of the deflectable region 125. Referring again to FIGS. 1A and 1B together, the catheter 120 further defines a lumen 127 extending therethrough from the proximal region 121 to the tip region 126. The proximal region 121 defines a proximal terminus (obscured by the handle 130 in FIGS. 1A and 1B; e.g., a proximal terminus 529 shown in FIG. 5A) of the catheter 120 that can be positioned within the handle 130, and the tip region 126 defines a distal terminus 128 of the catheter 120.

In some embodiments, the proximal region 121 has a first length, the intermediate region 122 has a second length less than the first length, and the distal region 123 has a third length less than the first and second lengths. For example, the first length can be between about 50-100 millimeters (e.g., about 80 millimeters), the second length can be between about 10-50 millimeters (e.g., about 25 millimeters), and the third length can be between about 1.0-10 millimeters (e.g., about 4.2 millimeters). In some embodiments, the transition region 124 can have a length of between about 0.1-5.0 millimeters (e.g., about 0.6 millimeters), the deflectable region 125 can have a length of between about 1.0-10 millimeters (e.g., about 3.0 millimeters), and the tip region 126 can have a length of between about 0.1-5.0 millimeters (e.g., about 0.6 millimeters). In other embodiments, the lengths of one or more of the regions 121-126 can be different. In some embodiments, the catheter 120 can have varying flexibilities, shapes, thicknesses, and/or other properties in/along the various regions 121-126.

In the illustrated embodiment, the handle 130 includes and/or is coupled to a valve 132. The valve 132 can include a branch or side port 133 configured to fluidly couple the lumen 127 of the catheter 120 to the tubing assembly 110, and can be integral with or coupled to the proximal region 121 of the catheter 120. In some embodiments, the valve 132 is a hemostasis valve that is configured to maintain hemostasis during a clot removal procedure by inhibiting or even preventing fluid flow in the proximal direction through the valve 132 as various components such as delivery sheaths, pull members, guidewires, interventional devices, other aspiration catheters, and so on are inserted through the valve 132 to be delivered through the catheter 120 to a treatment site in a blood vessel. In some embodiments, the valve 132 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety.

In the illustrated embodiment, the tubing assembly 110 fluidly couples the catheter 120 to a pressure source 102, such as a syringe. The tubing assembly 110 can include one or more tubing sections 112 (individually labeled as a first tubing section 112a and a second tubing section 112b), at least one fluid control device 114 (e.g., a valve), and at least one connector 116 (e.g., a Toomey tip connector) for fluidly coupling the tubing assembly 110 to the pressure source 102 and/or other suitable components. In some embodiments, the fluid control device 114 is a stopcock that is fluidly coupled to (i) the side port 133 of the valve 132 via the first tubing section 112a and (ii) the connector 116 via the second tubing section 112b. The fluid control device 114 is externally operable by a user to regulate the flow of fluid therethrough and, specifically, from the lumen 127 of the catheter 120 to the pressure source 102. In some embodiments, the connector 116 is a quick-release connector (e.g., a quick disconnect fitting) that enables rapid coupling/decoupling of the catheter 120 and the fluid control device 114 to/from the pressure source 102.

In the illustrated embodiment, the handle 130 includes a housing 134 and an actuator 136. The actuator 136 can be operably coupled to the catheter 120 and movable (e.g., rotatable) relative to the housing 134 to deflect (e.g., steer, flex) the deflectable region 125 from (i) a first position (e.g., an unflexed position, an aligned position) shown in FIG. 1A in which the deflectable region 125 is generally aligned with the intermediate region 122 and/or the proximal region 121 to (ii) a second position (e.g., a flexed position) shown in FIG. 1B in which the deflectable region 125 is deflected relative to the intermediate region 122 and/or the proximal region 121. That is, the actuator 136 can be configured to deflect the deflectable region 125 away from a longitudinal axis generally aligned with the proximal region 121 and/or the intermediate region 122. In some embodiments, the deflectable region 125 can have a bend angle Ain the second position (FIG. 1B) of greater than about 30 degrees, greater than about 50 degrees, greater than about 70 degrees, greater than about 90 degrees, or greater. In some embodiments, the bend angle A is about 90 degrees.

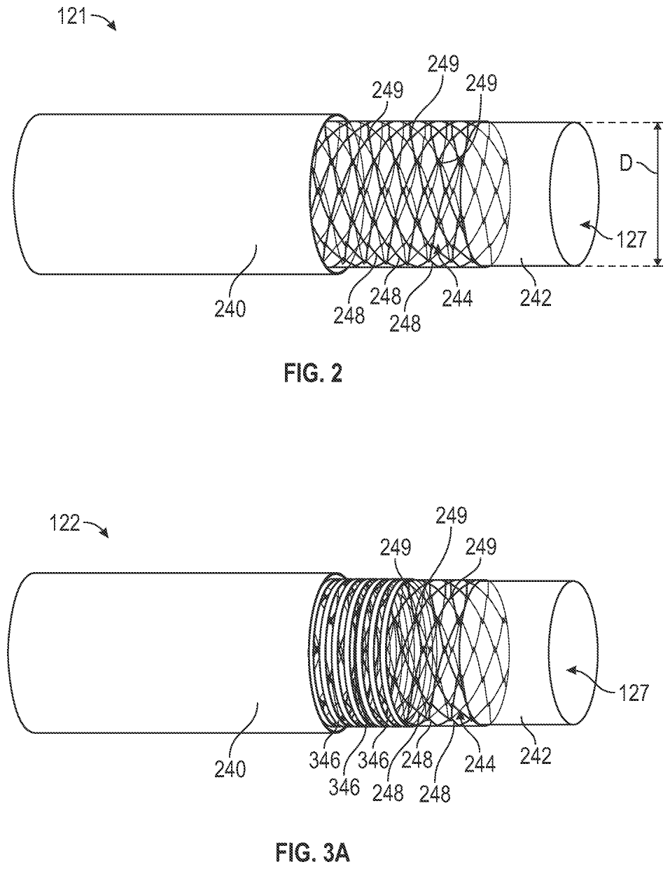

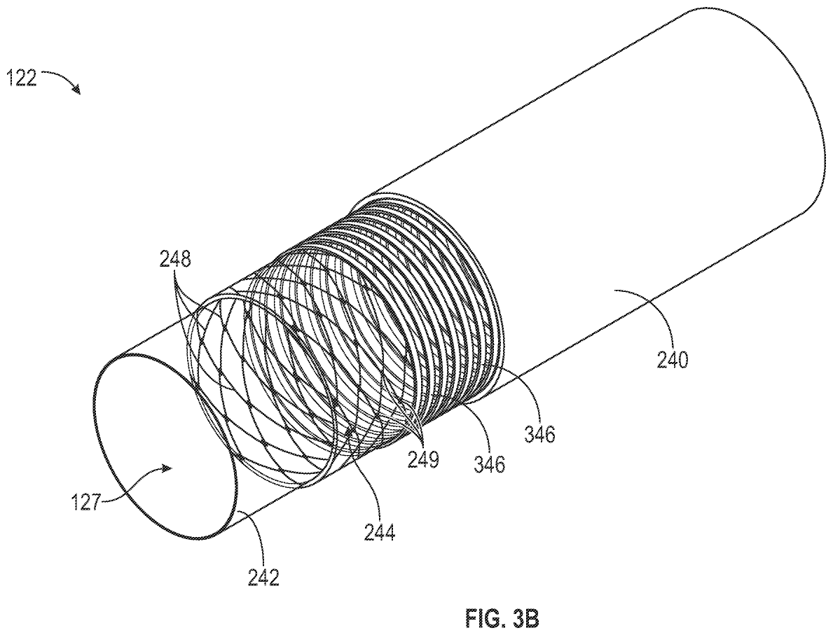

FIG. 2 is an enlarged, partially cut-away side view of a portion of the proximal region 121 of the catheter 120 in accordance with embodiments of the present technology. FIG. 3A is an enlarged, partially cut-away side view, and FIG. 3B is an enlarged, partially cut-away isometric view, of a portion of the intermediate region 122 of the catheter 120 in accordance with embodiments of the present technology. Referring to FIGS. 1-3B together, the catheter 120 includes an outer sheath 240 and an inner liner 242 extending through/defining each of the regions 121-126. The outer sheath 240 is positioned over (e.g., radially outside of) the inner liner 242. The outer sheath 240 can also be referred to as an outer jacket, an outer shaft, or an outer layer, and the inner liner 242 can also be referred to as an inner layer, an inner sheath, or an inner shaft.

In some embodiments, the outer sheath 240 can be formed from a plastic material, elastomeric material, and/or thermoplastic elastomer (TPE) material. In some embodiments, the outer sheath 240 can be formed from a TPE manufactured by Arkema S.A., of Colombes, France, such as the TPEs manufactured under the trademark “Pebax.” In some embodiments, the outer sheath 240 can have a varying hardness (e.g., durometer), thickness, flexibility, rigidity, and/or other property in one or more of the different regions 121-126. For example, the outer sheath 240 can have (i) a first hardness along the proximal region 121 of between about 65 D-75 D (e.g., about 72 D), (ii) a second hardness along the intermediate region 122 of between about 30 D-40 D (e.g., about 35 D), (iii) a third hardness along the transition region 124 of between about 50 D-60 D (e.g., about 55 D), (iv) a fourth hardness along the deflectable region of between about 20 D-30 D (e.g., about 25 D), and (v) a fifth hardness along the tip region 126 of between about 50 D-60 D (e.g., about 55 D). In other embodiments, the outer sheath 240 can have a different hardness or other property along one or more of the regions 121-126.

The inner liner 242 can be formed of a lubricious material that facilitates the movement (e.g., distal advancement, proximal retraction) of various components through the lumen 127, such as delivery sheaths, pull members, guidewires, interventional devices, other aspiration catheters, and the like. In some embodiments, the inner liner 242 can be formed from a polymer material, a fluoropolymer material (e.g., polytetrafluoroethylene (PTFE)), and/or another material having a high degree of lubricity. In some embodiments, the inner liner 242 can define a diameter D (FIG. 2) of the lumen 127, and the diameter D can be greater than about 6 French, greater than about 10 French, greater than about 16 French, greater than about 20 French, greater than about 24 French, or greater. In some embodiments, the diameter D is about 8 French, about 16 French, about 20 French, or about 24 French. In certain embodiments, the diameter D of the inner liner 242 is the same in each of the regions 121-126 while, in other embodiments, the diameter D can vary along one or more of the regions 121-126.

The catheter 120 can further include a braid 244 extending along the proximal region 121 and the intermediate region 122 between the outer sheath 240 and the inner liner 242. In some embodiments, the braid 244 terminates at or before the distal region 123 such that the braid 244 does not extend along the transition region 124, the deflectable region 125, or the tip region 126. In the illustrated embodiment, the catheter 120 further includes a coil 346 (FIGS. 3A and 3B) extending at least partially along the intermediate region 122 between the braid 244 and the outer sheath 240. In some embodiments, the coil 346 extends only along the intermediate region 122 and does not extend into the proximal region 121 or the distal region 123.

The braid 244 can include wires, filaments, threads, sutures, fibers, or the like (collectively “wires 248”) that have been woven or otherwise coupled, attached, formed, and/or joined together at a plurality of interstices 249. Accordingly, the braid 244 can also be referred to as a braided structure, a braided filament structure, a braided filament mesh structure, a mesh structure, a mesh filament structure, and the like. In some embodiments, the wires 248 can comprise metals, polymers, and/or composite materials. In some embodiments, individual ones of the wires 248 can be rolled flat wires having a cross-sectional dimension of between about 0.0005-0.005 inch (e.g., about 0.002 inch) by about 0.002-0.005 inch (e.g., about 0.0033 inch).

In the illustrated embodiment, the coil 346 is a single wire wound around the braid 244 and the inner liner 242 along the intermediate region 122. In other embodiments, the coil 346 can include more than one wire wound about the braid 244. For example, the coil 346 can include multiple wires wound over one another and/or multiple wires wound to at least partially overlap one another to form a braided or overlapping coil structure on the braid 244. In other embodiments, the coil 346 can be formed directly over the inner liner 242, and the braid 244 can be formed over the coil 346. The coil 346 can be formed from a metallic or other suitably strong material, such as nickel-titanium alloys (e.g. nitinol), platinum, cobalt-chrome alloys, stainless steel, tungsten, and/or titanium.

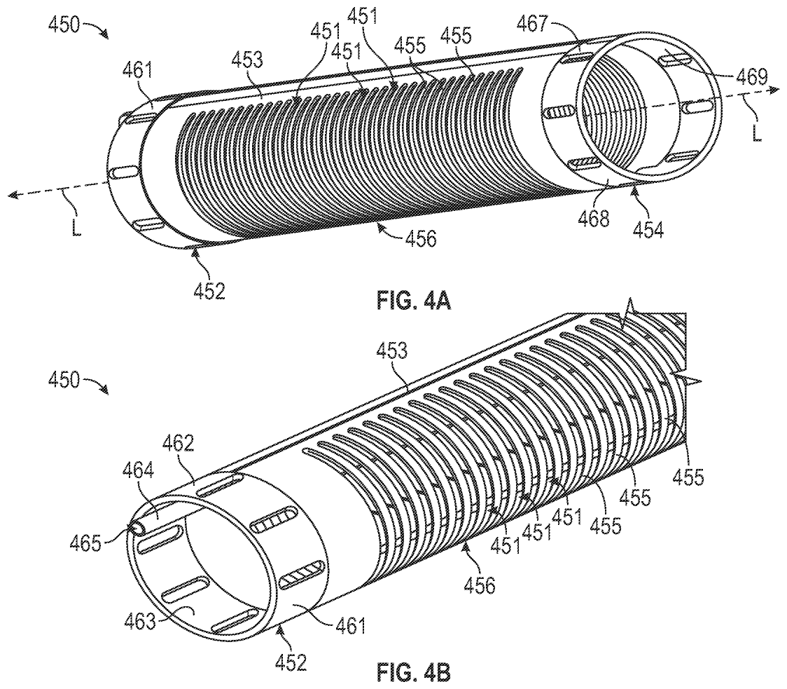

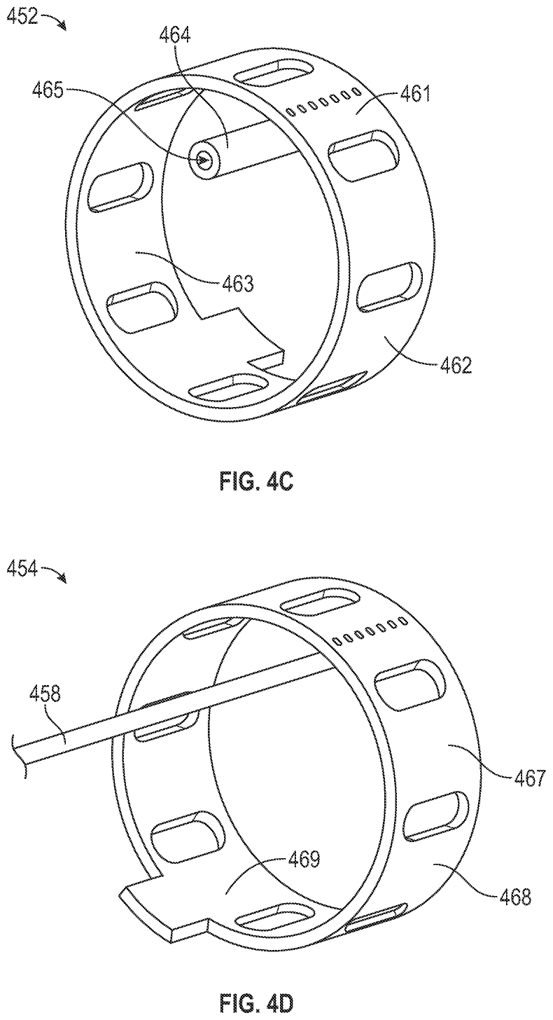

FIGS. 4A and 4B are a distally-facing isometric view and an enlarged proximally-facing isometric view, respectively, of a deflectable member 450 of the deflectable region 125 of the catheter 120 in accordance with embodiments of the present technology. The deflectable member 450 can be positioned between the outer sheath 240 and the inner liner 242 (FIGS. 2-3B), which are both omitted in FIGS. 4A and 4B for clarity. In the illustrated embodiment, the deflectable member 450 includes a proximal ring 452, a distal ring 454, and a tube portion 456 extending between the proximal ring 452 and the distal ring 454. The deflectable member 450 can be formed from a flexible metallic material—such as nickel-titanium alloys (e.g. nitinol), platinum, cobalt-chrome alloys, stainless steel, tungsten, and/or titanium—or another suitably strong and flexible material. Similarly, the deflectable member 450 can be manufactured (e.g., laser cut) as a single integral piece, or one or more of the proximal ring 452, the distal ring 454, and the tube portion 456 can be separately manufactured and then coupled (e.g., welded, tack welded, adhered, fastened) together.

FIGS. 4C and 4D are isometric views of the proximal ring 452 and the distal ring 454, respectively, in accordance with embodiments of the present technology. Referring to FIGS. 4A-4C together, the proximal ring 452 can include an annular body 462 having an outer surface 461 and an inner surface 463. An annular member 464 can be coupled (e.g., welded, tack welded, adhered, fastened) to the inner surface 463 of the annular member 464 and can define/include a lumen 465. Referring to FIGS. 4A and 4D together, the distal ring 454 can include an annular body 468 having an outer surface 467 and an inner surface 469. Referring to FIGS. 4A-4D together, the lumen 465 of the annular body 462 is configured to slidably receive a pull wire 458 (FIG. 4D). The pull wire 458 can be coupled (e.g., welded, tack welded, adhered, fastened) to the inner surface 469 of the distal ring 454 and can extend from the distal ring 454 to the handle 130 (FIG. 1), as described in greater detail below with reference to FIGS. 5A and 5B.

Referring again to FIGS. 4A and 4B together, the tube portion 456 can include a plurality of openings 451 extending partially about a circumference of the tube portion 456 to define a spine 453 and a plurality of ribs 455. In the illustrated embodiment, the spine 453 extends generally parallel to a longitudinal axis L (FIG. 4A) of the deflectable member 450 and is generally aligned with the annular member 464 and the pull wire 458 (FIG. 4D). That is, the pull wire 458 can extend through the tube portion 456 generally parallel to the spine 453. In some embodiments, the openings 451 are generally elongate openings that extend (i) generally parallel to one another and (ii) circumferentially about the longitudinal axis L such that, for example, the ribs 455 have a generally similar or identical shape. The openings 451 and/or the ribs 455 can all have the same dimensions as shown in FIGS. 4A and 4B while, in other embodiments, some or all of the openings 451 and/or the ribs 455 can have different dimensions and/or arrangements about the tube portion 456. In some embodiments, the tube portion 456 can be a laser-cut hypo tube.

Referring to FIGS. 2-4D together, in some embodiments the deflectable member 450 can be positioned between the outer sheath 240 and the inner liner 242 such that (i) the outer sheath 240 extends over/along the outer surface 461 of the proximal ring 452, an outer surface of the tube portion 456, and the outer surface 467 of the distal ring 454 and (ii) the inner liner 242 extends over/along the inner surface 463 of the proximal ring 452, an inner surface of the tube portion 456, and the inner surface 469 of the distal ring 454. In some embodiments, some or all of the pull wire 458 can be coated with PTFE or another suitable material (e.g., a fluoropolymer material). For example, the PTFE material can be omitted where the pull wire 458 is attached to the distal ring 454. The PTFE or other coating material can help inhibit the outer sheath 240 from adhering to the pull wire 458—thereby allowing the pull wire 458 to be moved relative to the deflectable member 450 after the outer sheath 240 is applied thereover.

Referring to FIGS. 1-3B together, in some embodiments the transition region 124 and the tip region 126 can include only the outer sheath 240 and the inner liner 242. In some embodiments, the transition region 124 and/or the tip region 126 can include a marker band (not shown), such as a radiopaque marker configured to facilitate visualization of the position of the catheter 120 during a medical procedure (e.g., a clot removal procedure) using the catheter 120. For example, the transition region 124 and the tip region 126 can each include a radiopaque marker to facilitate visualization of the deflectable region 125 of the catheter 120.

Referring to FIGS. 1-4D together, in some embodiments, the catheter 120 can be formed about a mandrel or other elongate member. For example, the inner liner 232 can first be positioned about the mandrel. Then, the braid 244 can be formed (e.g., wound, braided) about the inner liner 242 around the mandrel (e.g., along the proximal and intermediate regions 121, 122) and/or the deflectable member 450 can be positioned about the inner liner 242 around the mandrel (e.g., along the deflectable region 125). Next, the coil 346 can be wound around the mandrel about the braid 244 over the intermediate region 122. Next, the outer sheath 240 can be positioned over the inner liner 242, the braid 244, the coil 346, and the deflectable member 450, and then heat shrunk or otherwise secured thereto. In some embodiments, the outer sheath 240 can be fused to the inner liner 242, the braid 244, the coil 346, and/or the deflectable member 450 to secure these components of the catheter 120 together.

FIG. 5A is a partially cross-sectional side view of the handle 130 and a portion of the proximal region 121 of the catheter 120 in accordance with embodiments of the present technology. FIG. 5B is an enlarged cross-sectional isometric view of a portion of the handle 130 shown in FIG. 5A. Referring to FIGS. 5A and 5B, together, the housing 134 defines a proximal chamber 570 (e.g., a volume, lumen, compartment) and a distal chamber 572 that can be separated by the actuator 136. The valve 132 can be coupled to the housing 134 (e.g., a proximal portion of the housing 134) and positioned at least partially within the proximal chamber 570.

In the illustrated embodiment, the handle 130 includes a hollow tube member 574 positioned at least partially within the proximal chamber 570. The tube member 574 can include a proximal end portion 571a and a distal end portion 571b coupled to (e.g., secured to) the actuator 136. The tube member 574 can define a lumen 573 extending between the proximal and distal end portions 571a-b, and the tube member 574 can have a threaded inner surface 575 extending at least partially along the lumen 573. The actuator 136 can be a rotatable member, such as a wheel, grip wheel, or dial that is rotatable relative to the housing 134 to rotate the tube member 574 within the proximal chamber 570.

In the illustrated embodiment, the handle 130 further includes a catheter support or guide 576 extending at least partially through (i) the distal chamber 572, (ii) the actuator 136 (e.g., through a lumen in the actuator), (iii) the lumen 573 of the tube member 574, and (iv) the proximal chamber 570. In some embodiments, the catheter guide 576 defines a lumen 577 extending therethrough and includes a proximal flange portion 578 that can be secured to the housing 134. In some embodiments, the catheter guide 576 is fixed to the housing 134 such that the catheter guide 576 does not rotate when the actuator 136 is actuated to move the tube member 574. The proximal region 121 of the catheter 120 can extend into the handle 130, through the lumen 577 in the catheter guide 576, and to the valve 132. The proximal terminus 529 of the catheter 120 can be fluidly coupled to the valve 132. Accordingly, the catheter 120, the catheter guide 576, and the tube member 574 can be coaxially aligned. In other embodiments, the catheter guide 576 can be omitted.

The handle 130 can further include a shuttle member 580 positioned at least partially in the lumen 573 of the tube member 574 over the catheter guide 576 (e.g., over an outer surface thereof). In some embodiments, the shuttle member 580 is a hollow member slidably positioned over the catheter guide 576 and movable relative to the catheter 120. In the illustrated embodiment, the shuttle member 580 includes a threaded portion 582 having a threaded outer surface 583 and an anchor portion 584 extending from the threaded portion 582. The threaded outer surface 583 is configured to engage the threaded inner surface 575 of the tube member 574 such that, for example, movement of the tube member 574 drives the shuttle member 580 to move through the lumen 573 over the catheter guide 576 and relative to the catheter 120.

In the illustrated embodiment, the pull wire 458 extends along the catheter 120 into the handle 130 where it secured to the anchor portion 584 of the shuttle member 580. More specifically, the pull wire 458 can extend from the distal ring 454 of the deflectable member 450 (FIG. 4D) and through/along the transition, intermediate, and proximal regions 124, 122, 121 of the catheter 120 (FIGS. 1A and 1B) to the handle 130. For example, the pull wire 458 can be routed (i) through a lumen formed in the wall of the catheter 120 or (ii) simply between the outer sheath 240 and inner liner 242 (FIGS. 2-3B). In the illustrated embodiment, the pull wire 458 exits the catheter 120 and the catheter guide 476 (e.g., via openings therein) and enters the distal chamber 572. From the distal chamber 572, the pull wire 458 can extend through the actuator 136 and through the lumen 573 of the tube member 574 to the anchor portion 584. As best seen in FIG. 5B, in some embodiments the pull wire 458 can be secured to anchor portion 584 via a screw 581 or other fastener. In some embodiments, the handle 130 can further include a biasing member 585, such as a coil spring, coupled to and/or over the pull wire 458. The biasing member 585 can be configured to smooth/distribute tension loads on the pull wire 458 during operation that might otherwise damage the pull wire 458 and/or various components of the handle 130.

Referring to FIGS. 1A, 1B, and 4A-5B together, the deflectable region 125 (and correspondingly the deflectable member 450) is in the first position and the handle 130 is in a corresponding first position in which the shuttle member 580 is positioned distally within the lumen 473 of the tube member 574 proximate to the actuator 136 and/or the distal end portion 571b of the tube member 574. To move the deflectable region 125 to the second (e.g., bent) position, a user can rotate the actuator 136 in a first direction to rotate the tube member 574. The rotation of the tube member 574 can drive the shuttle member 580 to move proximally through the lumen 573 in a direction toward the proximal end portion 571a of the tube member 574 via the engagement of the threaded outer surface 583 with the threaded inner surface 575. That is, the handle 130 is configured to translate the rotational movement of the actuator 136 into linear movement of the shuttle member 580. As the shuttle member 580 moves proximally, the shuttle member 580 pulls the pull wire 458 proximally and increases the tension therein. The pull wire 458 thus moves (e.g., slides) proximally through the lumen 465 in the annular member 464 of the deflectable member 450 and, because the pull wire 458 is fixedly attached to the distal ring 454 of the deflectable member 450, the pull wire 458 urges the distal ring 454 proximally relative to the proximal ring 452. This differential force causes the tube portion 456 of the deflectable member 450 to bend toward the second position shown in FIG. 1B. More specifically, because the pull wire 458 is aligned with the spine 453 of the deflectable member 450, the spine 453 can define an inner radius of the bend while the ribs 455 flex away from one another, thereby increasing a size of the openings 451. To return the deflectable region 125 from the second position to the first position, the user can rotate the actuator 136 in a second direction opposite the first direction to translate the shuttle member 580 distally through the lumen 573 to decrease the tension in the pull wire 458, thereby allowing the deflectable member 450 to return to the relaxed position shown in FIGS. 4A and 4B.

In other embodiments, the handle 130 can include other features for moving/driving the shuttle member 580 through the housing 134 to tension the pull wire 458. For example, the actuator 136 can be a slider, clip, or other actuator movable relative to the housing 134.

Referring to FIGS. 1A-5B together, in some aspects of the present technology, the catheter 120 is configured to be steered to and positioned in difficult-to-reach regions of the anatomy of a patient while still having a relatively large size (e.g., 20 French, 24 French, greater than 24 French). More particularly, the catheter 120 can have an improved torque response and flexibility compared to conventional catheters having the same size. For example, the braid 234 can provide good torque response along the proximal and intermediate regions 121, 122 of the catheter 120. Moreover, the varying hardness (e.g., distally decreasing hardness) of the outer sheath 240 can provide (i) good torque response and/or pushability at the proximal region 121 and (ii) increased flexibility at the intermediate and distal regions 122, 123. Additionally, the deflectable region 125 is configured (e.g., shaped, sized) to be positioned within and steered/flexed into the difficult-to-reach regions of the anatomy. Further, the coil 346 can provide increased hoop strength at the intermediate region 122 while still allowing the catheter 120 to flex. For example, the coil 346 can inhibit or even prevent kinking or other unwanted movement of the catheter 120 when the lumen 127 is aspirated during a clot removal procedure.

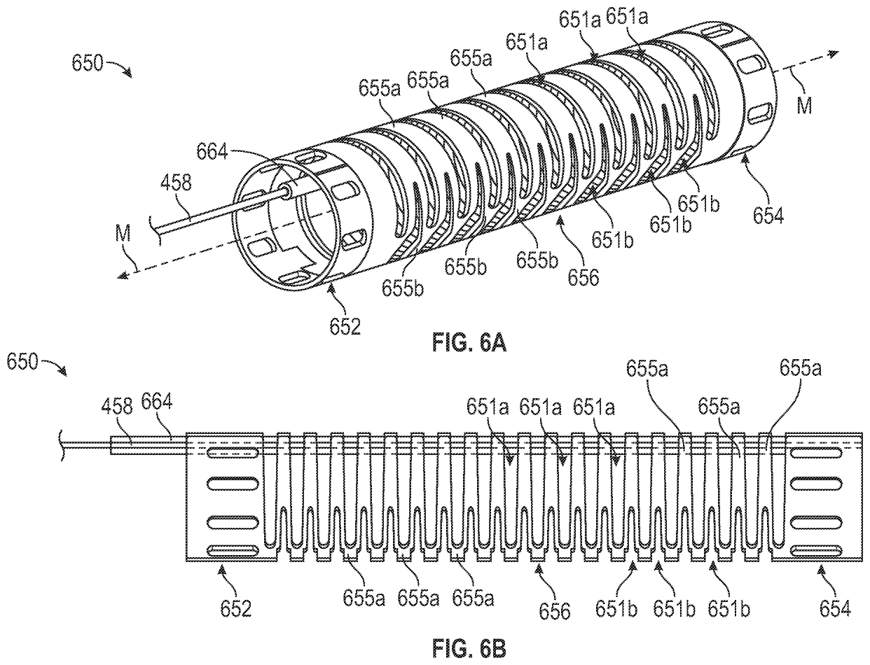

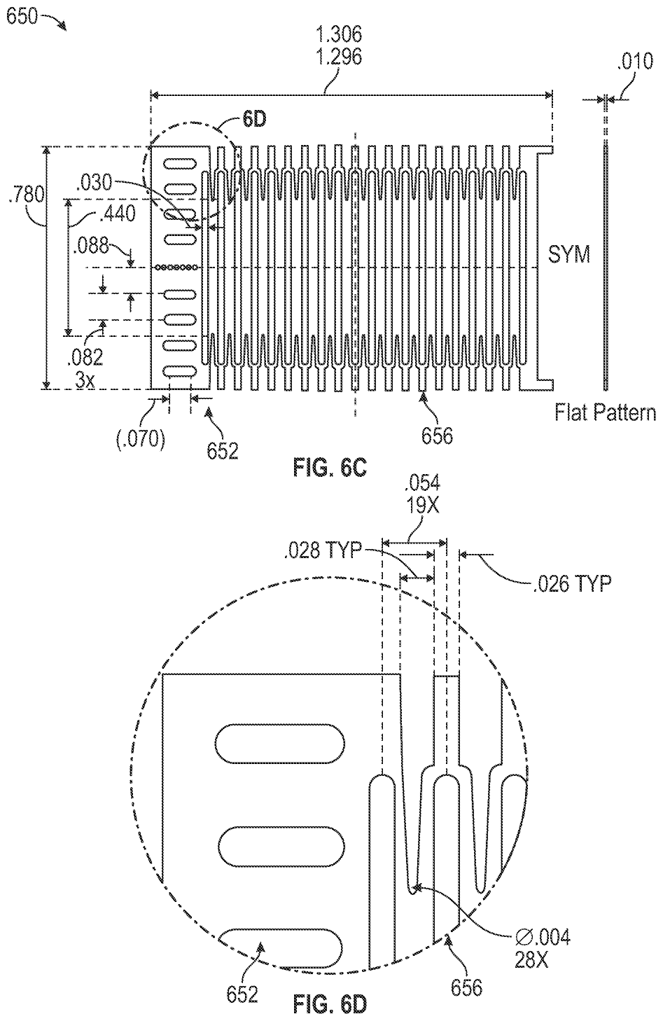

FIGS. 6A and 6B are a distally-facing isometric view and a side view, respectively, of a deflectable member 650 in accordance with additional embodiments of the present technology. The deflectable member 650 is configured to be positioned in the deflectable region 125 of the catheter 120 (FIG. 1) and can include some features generally similar or identical to the deflectable member 450 described in detail above with reference to FIGS. 4A-4D. For example, in the illustrated embodiment the deflectable member 650 includes a proximal ring 652, a distal ring 654, and a tube portion 656 extending between the proximal ring 652 and the distal ring 654. The proximal ring 652 includes an annular member 664 coupled thereto and configured to slidably receive the pull wire 458. The pull wire 458 can extend through the tube portion 656 and be fixedly secured (e.g., welded) to the distal ring 654.

In the illustrated embodiment, the tube portion 656 includes a plurality of openings 651 (identified individually as first openings 651a and second openings 651b) extending partially about a circumference of the tube portion 656 to define a plurality of ribs 655 (identified individually as first ribs 655a and second ribs 655b). In some embodiments, the first openings 651a are generally elongate openings that extend (i) generally parallel to one another and (ii) circumferentially about a longitudinal axis M of the deflectable member 650 such that, for example, the first ribs 655a have a generally similar or identical shape. Similarly, the second openings 651b can each have an elongate tapered shape and can extend (i) generally parallel to one another and (ii) circumferentially about the longitudinal axis M of the deflectable member 650 such that, for example, the second ribs 655b have a generally similar or identical shape. In the illustrated embodiment, the second ribs 655b have a smaller dimension (e.g. width) in a direction along the longitudinal axis M than the first ribs 655a. Accordingly, the second ribs 655b can be relatively more flexible than the first ribs 655a.

In some embodiments, the pull wire 458 can extend over/adjacent to the first ribs 655a. Accordingly, referring to FIGS. 5-6B together, actuation of the actuator can 136 pull the pull wire 458 to urge the distal ring 654 proximally relative to the proximal ring 652. This differential force causes the tube portion 656 of the deflectable member 650 to bend such that, for example, a portion of the first ribs 655a define an inner radius of the bend while the second ribs 655b flex away from one another, thereby increasing a size of the second openings 651b (e.g., and conversely decreasing a size of the first openings 651a).

In some embodiments, all or a portion of the deflectable member 650 can be manufactured as a single integral piece. For example, FIG. 6C is top view of flat pattern that can be cut to integrally form the proximal ring 652 and the tube portion 656 of the deflectable member 650 in accordance with embodiments of the present technology. FIG. 6D is an enlarged top view of a portion of the pattern shown in FIG. 6C. Referring to FIGS. 6C and 6D together, the pattern can be laser cut from a single piece of material (e.g., stainless steel), formed to have the three-dimensional tubular shape shown in FIGS. 6A and 6B, and then welded or otherwise adhered together to form the deflectable member 650.

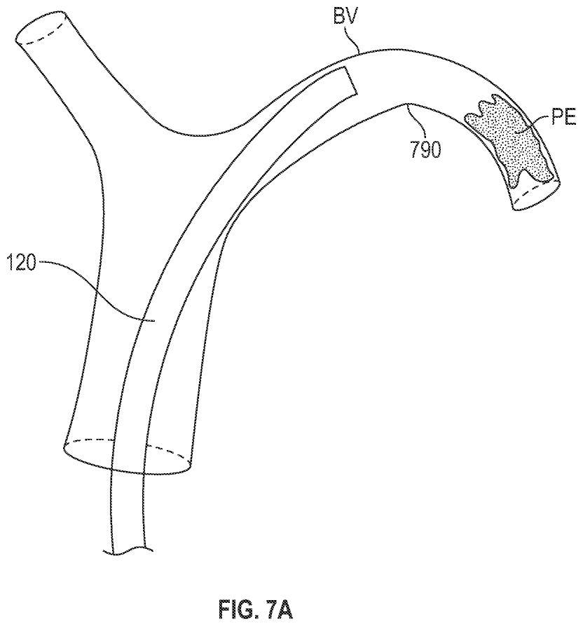

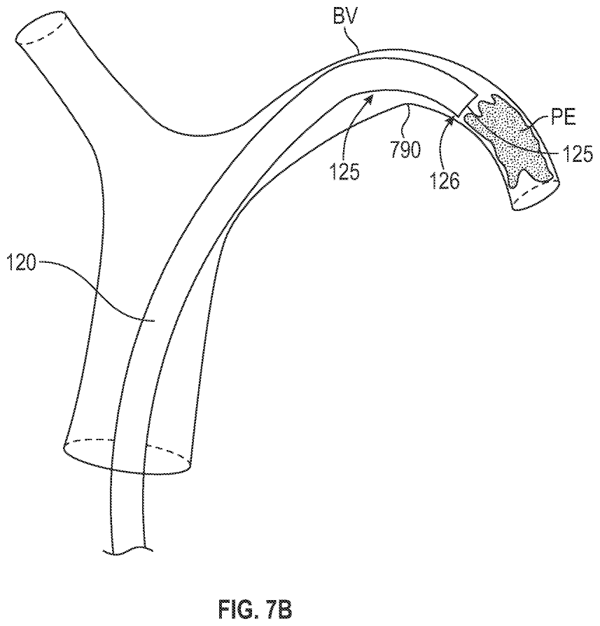

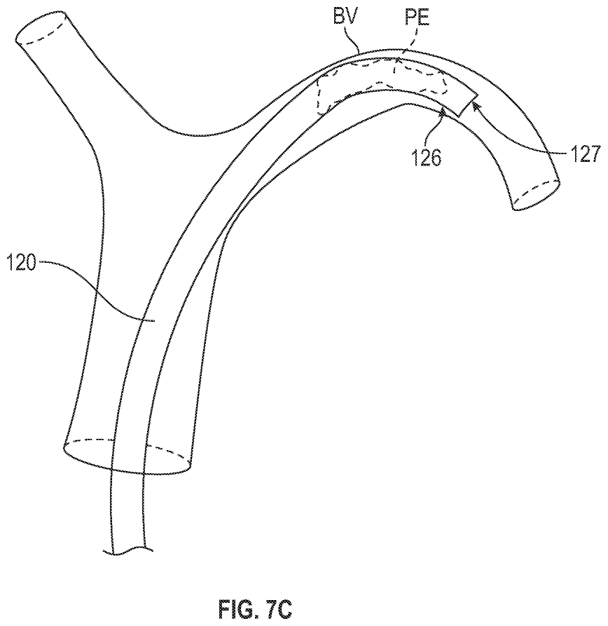

FIGS. 7A-7C are side views of a portion of the catheter 120 of the clot removal system 100 during a procedure for removing clot material PE (e.g., a pulmonary embolism) from within a blood vessel BV (e.g., a pulmonary blood vessel) of a patient (e.g., a human patient) in accordance with embodiments of the present technology. As noted above, in some embodiments the clot removal procedure illustrated in FIGS. 7A-7C can be generally similar or identical to any of the clot removal procedures disclosed in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety.

With reference to FIGS. 1A-7A together, the catheter 120 can be advanced through the patient toward and/or proximate to the clot material PE within the blood vessel BV (e.g., advanced to a treatment site within the blood vessel BV). In some embodiments, however, the blood vessel BV can include a hard-to-reach (e.g., tortuous) region, such as a region beyond a bend 790 in the blood vessel BV that can have a relatively small radius of curvature. The region of the blood vessel BV distal of the bend 790 can be difficult to reach due to the required approach angle, varying anatomy of the blood vessel BV, and/or irregularities due to illness of the patient.

Accordingly, with reference to FIGS. 1A-5B and 7B together, the deflectable region 125 can be moved fully or partially from the first position (FIG. 1A) to the second position (FIG. 1B) before and/or during further advancement of the catheter 120 toward the clot material PE. More specifically, the user can actuate (e.g., rotate) the actuator 136 of the handle to pull the pull wire 458 to deflect the deflectable member 450 to deflect the deflectable region 125, as described in detail above. In some embodiments, the catheter 120 can be advanced through the blood vessel BV until the distal terminus 128 of the catheter 120 is positioned proximate to a proximal portion of the clot material PE. In some embodiments, the position of the distal terminus 128 can be confirmed or located via visualization of a marker band (not shown; e.g., in/along the tip region 126) using fluoroscopy or another imaging procedure (e.g., a radiographic procedure). In other embodiments, the distal terminus 128 can be positioned at least partially within the clot material PE or distal of the clot material PE.

In some aspects of the present technology, moving the deflectable region 125 to the second position helps the catheter 120 flex/bend around the bend 790 and into the hard-to-reach region of the blood vessel BV distal thereof. In some embodiments, before advancing the catheter 120 to the position shown in FIG. 7B, the catheter 120 can be rotated to align the deflectable region 125 with the bend 790. In contrast, conventional catheters of the same size may be too stiff to easily position proximate the clot material PE. For example, such conventional catheters may “rainbow” over the clot material PE by following or tracking along the wall of the blood vessel BV at the outside of the bend 790. In addition to the deflectable region 125, both (i) the varying hardness of the outer sheath 240 (FIGS. 2-3B) and (ii) the flexibility of the braid 244 (FIGS. 2-3B) and the coil 346 (FIGS. 3B and 3C) can help the catheter 120 flex through the anatomy of the blood vessel BV to the desired position proximate the clot material PE.

Access to the pulmonary vessels can be achieved through the patient's vasculature, for example, via the femoral vein. In some embodiments, the clot removal system 100 can include an introducer (e.g., a Y-connector with a hemostasis valve; not shown) that can be partially inserted into the femoral vein. A guidewire (not shown) can be guided into the femoral vein through the introducer and navigated through the right atrium, the tricuspid valve, the right ventricle, the pulmonary valve, and into the main pulmonary artery. Depending on the location of the clot material PE, the guidewire can be guided to one or more of the branches of the right pulmonary artery and/or the left pulmonary artery. In some embodiments, the guidewire can be extended entirely or partially through the clot material PE. In other embodiments, the guidewire can be extended to a location just proximal of the clot material PE. After positioning the guidewire, the catheter 120 can be placed over the guidewire and advanced to the position proximate to the clot material PE as illustrated in FIG. 7B. In some embodiments, the guidewire can then be withdrawn while, in other embodiments, the guidewire can remain and can be used to guide other catheters (e.g., delivery catheters, additional aspiration guide catheters, etc.), interventional devices, etc., to the treatment site. It will be understood, however, that other access locations into the venous circulatory system of a patient are possible and consistent with the present technology. For example, the user can gain access through the jugular vein, the sub clavian vein, the brachial vein, or any other vein that connects or eventually leads to the superior vena cava. Use of other vessels that are closer to the right atrium of the patient's heart can also be advantageous as it reduces the length of the instruments needed to reach the clot material PE.

With reference to FIGS. 1A, 1B, and 7C together, the pressure source 102 is configured to generate (e.g., form, create, charge, build-up) a vacuum (e.g., negative pressure) and store the vacuum for subsequent application to the catheter 120. For example, after positioning the catheter 120 proximate the clot material PE, a user can first close the fluid control device 114 before generating the vacuum in the pressure source 102 by, for example, withdrawing the plunger of a syringe coupled to the connector 116. In this manner, a vacuum is charged within the pressure source 102 (e.g., a negative pressure is maintained) before the pressure source 102 is fluidly connected to the lumen 127 of the catheter 120. To aspirate the lumen 127 of the catheter 120, the user can open the fluid control device 114 to fluidly connect the pressure source 102 to the catheter 120 and thereby apply or release the vacuum stored in the pressure source 102 to the lumen 127 of the catheter 120.

Opening of the fluid control device 114 instantaneously or nearly instantaneously applies the stored vacuum pressure to the tubing assembly 110 and the catheter 120, thereby generating a suction pulse throughout the catheter 120. In particular, the suction is applied at the tip region 126 of the catheter 120 to suck/aspirate at least a portion of the clot material PE into the lumen 127 of the catheter 120, as shown in FIG. 7C. In one aspect of the present technology, pre-charging or storing the vacuum in the pressure source 102 before applying the vacuum to the lumen 127 of the catheter 120 is expected to generate greater suction forces and corresponding fluid flow velocities at and/or near the tip region 126 of the catheter 120 compared to simply activating the pressure source 102 while it is fluidly connected to the catheter 120.

Sometimes, as shown in FIG. 7C, discharging the vacuum stored in the pressure source to aspirate the lumen 127 of the catheter 120 may remove substantially all (e.g., a desired amount) of the clot material PE from the blood vessel BV. That is, a single aspiration pulse may adequately remove the clot material PE from the blood vessel BV. In other embodiments, a portion of the clot material PE may remain in the blood vessel BV. In such instances, the user may wish to again apply vacuum pressure (conduct an “aspiration pass”) to remove all or a portion of the remaining clot material PE in the blood vessel BV. In such instances, the pressure source 102 can be disconnected from the tubing assembly 110 and drained (e.g., aspirated clot removal removed) before the pressure source 102 is reconnected to the tubing assembly 110 and activated once again. After removing a desired amount of the clot material PE, the catheter 120 can be withdrawn from the patient.

In some aspects of the present technology, the relatively great flexibility and torquability of the catheter 120 allow the catheter 120 to be positioned in difficult-to-reach areas of the blood vessel BV (or elsewhere in the vasculature of the patient) without decreasing the size of the lumen 127 and while keeping the lumen 127 of constant diameter throughout. It is expected that the increased size of the lumen 127 will provide greater suction forces over a smaller period of time (e.g., will provide a larger vacuum impulse). In some embodiments, the greater suction forces can facilitate the removal of clot material from a blood vessel of a patient even where the clot material is strongly lodged or attached within the blood vessel (e.g., a chronic clot). Accordingly, in contrast to conventional catheters, the catheter 120 can be used to generate greater aspirational forces for improved clot removal in hard-to-reach places of the vasculature. In additional aspects of the present technology, the coil 336 (FIGS. 3B and 3C) can provide a high hoop strength that inhibits or even prevents kinking or other unwanted movement of the catheter 120 when the pressure source 102 is used to generate a suction pulse at the distal region 123 of the catheter 120.

Although described in the context of removing clot material from pulmonary blood vessels, in other embodiments the clot removal system 100 can be used to remove clot from other locations in the body of the patient. For example, the clot removal system 100 can used to aspirate or otherwise remove clot material (e.g., stationary or in transit) and/or vegetation from the heart (e.g., the right atrium, tricuspid valve, pulmonary valve), the vena cava, the renal arteries, and so on.

Several aspects of the present technology are set forth in the following examples:

1. An aspiration catheter, comprising:
a proximal region; and
a distal region including a deflectable member, wherein the deflectable member includes—
   a proximal ring;
   a distal ring configured to be fixedly attached to a pull wire; and
   a tube portion extending between the proximal and distal rings, wherein the tube portion includes a plurality of openings extending therethrough to define a plurality of ribs, and wherein the ribs are configured to flex away from each other when the pull wire is pulled proximally.

2. The aspiration guide catheter of example 1 wherein the tube portion includes a spine extending in a direction between the proximal and distal rings, wherein the ribs extend away from the spine, and wherein the spine is configured to extend generally parallel to and over the pull wire.

3. The aspiration catheter of example 1 or example 2 wherein the proximal region and the distal region define a lumen having a diameter of 20 French or greater.

4. The aspiration catheter of any one of examples 1-3, further comprising an intermediate region between the proximal and distal regions, wherein the proximal region and the intermediate region include a braid of wires extending therethrough.

5. The aspiration catheter of example 4 wherein the intermediate region includes a wire coiled around the braid.

6. The aspiration catheter of any one of examples 1-5 wherein the tube portion extends along a longitudinal axis in a relaxed state, and wherein the openings extend circumferentially about the longitudinal axis and generally parallel to one another in the relaxed state.

7. The aspiration catheter of any one of examples 1-6 wherein the proximal ring includes an annular member configured to slidably receive the pull wire therethrough.

8. A clot removal system, comprising:
an aspiration catheter including a proximal region and a distal region, wherein the distal region includes a deflectable member;
a handle coupled to the proximal region of the aspiration catheter, wherein the handle includes an actuator; and
a pull wire extending between the actuator and the deflectable member, wherein actuation of the actuator is configured to pull the pull wire to deflect the deflectable member to deflect the distal region of the aspiration catheter relative to the proximal region.

9. The clot removal system of example 8 wherein the aspiration catheter extends along an axis, and wherein the actuation of the actuator is configured to deflect the distal region of the aspiration catheter away from the axis by about 90 degrees or greater.

10. The clot removal system of example 8 or example 9 wherein the aspiration guide catheter has a size of 20 French or greater.

11. The clot removal system of any one of examples 8-10 wherein the deflectable member has a tubular shape that extends along a longitudinal axis, and wherein the deflectable member includes (a) a spine extending parallel to the longitudinal axis and (b) a plurality of ribs extending from the spine and circumferentially about the longitudinal axis.

12. The clot removal system of example 11 wherein the deflectable member has a distal portion and a proximal portion, and wherein the pull wire is attached to the distal portion of the deflectable member.

13. The clot removal system of example 12 wherein the actuation of the actuator is configured pull the distal portion proximally relative to the proximal portion.

14. The clot removal system of example 12 or example 13 wherein the ribs define a plurality of openings therebetween, and wherein the actuation of the actuator is configured pull the distal portion of the deflectable member proximally relative to the proximal portion to bend the spine and increase a size of the openings.

15. The clot removal system of any one of examples 8-14 wherein the aspiration catheter further includes—
an intermediate region between the proximal and distal regions;
an inner liner extending through the proximal, intermediate, and distal regions;
a braid of wires extending through the proximal and intermediate regions over the inner liner;
a wire extending through the intermediate region and coiled around the braid; and
an outer liner extending through the proximal, intermediate, and distal regions over the inner liner, the braid, and the wire, wherein the deflectable member is positioned between the inner and outer liners in the distal region.

16. The clot removal system of example 15 wherein—
the distal region further includes a proximal transition region, a distal tip region, and a deflectable region between the proximal transition region and the distal tip region;
the deflectable member is positioned in the deflectable region;
the outer liner has a first hardness in the proximal transition region, a second hardness in the deflectable region, and a third hardness in the distal tip region; and
the second hardness is less than the first hardness and less than the third hardness.

17. A method of removing clot material from a blood vessel, the method comprising:
advancing an aspiration catheter through the blood vessel, wherein the aspiration catheter includes a distal portion and a proximal portion;
actuating a handle coupled to the aspiration catheter to deflect the distal portion of the aspiration catheter away from a longitudinal axis of the proximal portion;
positioning a distal tip of the aspiration catheter proximate to the clot material;
activating a pressure source coupled to the aspiration catheter via a fluid control device, while the fluid control device is closed, to generate a vacuum in the pressure source; and
opening the fluid control device to apply the vacuum to the aspiration catheter to thereby aspirate at least a portion of the clot material into the aspiration catheter.

18. The method of example 17 wherein actuating the handle to deflect the distal portion of the aspiration catheter includes deflecting the distal portion of the aspiration catheter away from the longitudinal axis in a deflection direction, and wherein the method further comprises rotating the aspiration catheter such that the deflection direction is at least partially aligned with a bend in the blood vessel.

19. The method of example 17 or example 18 wherein the aspiration catheter has a size of 20 French or greater.

20. The method of any one of examples 17-19 wherein the aspiration catheter includes a deflectable member positioned in the distal portion, and wherein actuating the handle includes rotating an actuator of the handle to pull a pull wire coupled to the deflectable portion proximally to deflect the deflectable member to deflect the distal portion of the aspiration catheter.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An aspiration catheter, comprising:
a proximal region; and
a distal region including a deflectable member, wherein the deflectable member includes—
a proximal ring;
a distal ring configured to be fixedly attached to a pull wire; and
a tube portion extending between the proximal and distal rings, wherein the tube portion includes a plurality of openings extending therethrough to define a plurality of ribs, and wherein the ribs are configured to flex away from each other when the pull wire is pulled proximally.

2. The aspiration guide catheter of claim 1 wherein the tube portion includes a spine extending in a direction between the proximal and distal rings, wherein the ribs extend away from the spine, and wherein the spine is configured to extend generally parallel to and over the pull wire.

3. The aspiration catheter of claim 1 wherein the proximal region and the distal region define a lumen having a diameter of 20 French or greater.

4. The aspiration catheter of claim 1, further comprising an intermediate region between the proximal and distal regions, wherein the proximal region and the intermediate region include a braid of wires extending therethrough.

5. The aspiration catheter of claim 4 wherein the intermediate region includes a wire coiled around the braid.

6. The aspiration catheter of claim 1 wherein the tube portion extends along a longitudinal axis in a relaxed state, and wherein the openings extend circumferentially about the longitudinal axis and generally parallel to one another in the relaxed state.

7. The aspiration catheter of claim 1 wherein the proximal ring includes an annular member configured to slidably receive the pull wire therethrough.

8. A clot removal system, comprising:
an aspiration catheter including a proximal region and a distal region, wherein the distal region includes a deflectable member;
a handle coupled to the proximal region of the aspiration catheter, wherein the handle includes an actuator; and
a pull wire extending between the actuator and the deflectable member, wherein actuation of the actuator is configured to pull the pull wire to deflect the deflectable member to deflect the distal region of the aspiration catheter relative to the proximal region.

9. The clot removal system of claim 8 wherein the aspiration catheter extends along an axis, and wherein the actuation of the actuator is configured to deflect the distal region of the aspiration catheter away from the axis by about 90 degrees or greater.

10. The clot removal system of claim 8 wherein the aspiration guide catheter has a size of 20 French or greater.

11. The clot removal system of claim 8 wherein the deflectable member has a tubular shape that extends along a longitudinal axis, and wherein the deflectable member includes (a) a spine extending parallel to the longitudinal axis and (b) a plurality of ribs extending from the spine and circumferentially about the longitudinal axis.

12. The clot removal system of claim 11 wherein the deflectable member has a distal portion and a proximal portion, and wherein the pull wire is attached to the distal portion of the deflectable member.

13. The clot removal system of claim 12 wherein the actuation of the actuator is configured pull the distal portion proximally relative to the proximal portion.

14. The clot removal system of claim 12 wherein the ribs define a plurality of openings therebetween, and wherein the actuation of the actuator is configured pull the distal portion of the deflectable member proximally relative to the proximal portion to bend the spine and increase a size of the openings.

15. The clot removal system of claim 8 wherein the aspiration catheter further includes—
an intermediate region between the proximal and distal regions;
an inner liner extending through the proximal, intermediate, and distal regions;
a braid of wires extending through the proximal and intermediate regions over the inner liner;
a wire extending through the intermediate region and coiled around the braid; and
an outer liner extending through the proximal, intermediate, and distal regions over the inner liner, the braid, and the wire, wherein the deflectable member is positioned between the inner and outer liners in the distal region.

16. The clot removal system of claim 15 wherein—
the distal region further includes a proximal transition region, a distal tip region, and a deflectable region between the proximal transition region and the distal tip region;
the deflectable member is positioned in the deflectable region;
the outer liner has a first hardness in the proximal transition region, a second hardness in the deflectable region, and a third hardness in the distal tip region; and
the second hardness is less than the first hardness and less than the third hardness.

17. A method of removing clot material from a blood vessel, the method comprising:
advancing an aspiration catheter through the blood vessel, wherein the aspiration catheter includes a distal portion and a proximal portion;
actuating a handle coupled to the aspiration catheter to deflect the distal portion of the aspiration catheter away from a longitudinal axis of the proximal portion;
positioning a distal tip of the aspiration catheter proximate to the clot material;
activating a pressure source coupled to the aspiration catheter via a fluid control device, while the fluid control device is closed, to generate a vacuum in the pressure source; and
opening the fluid control device to apply the vacuum to the aspiration catheter to thereby aspirate at least a portion of the clot material into the aspiration catheter.

18. The method of claim 17 wherein actuating the handle to deflect the distal portion of the aspiration catheter includes deflecting the distal portion of the aspiration catheter away from the longitudinal axis in a deflection direction, and wherein the method further comprises rotating the aspiration catheter such that the deflection direction is at least partially aligned with a bend in the blood vessel.

19. The method of claim 17 wherein the aspiration catheter has a size of 20 French or greater.

20. The method of claim 17 wherein the aspiration catheter includes a deflectable member positioned in the distal portion, and wherein actuating the handle includes rotating an actuator of the handle to pull a pull wire coupled to the deflectable portion proximally to deflect the deflectable member to deflect the distal portion of the aspiration catheter.
